(19) United States
(12) Patent Application Publication
(10) Pub. No.: US 2012/0053553 A1
(43) Pub. Date: Mar. 1, 2012

(54) ADAPTIVE CLOTHING BOXER SHORT UNDERGARMENT

(76) Inventor: Michell Renae Griggs, Lubbock, TX (US)

(21) Appl. No.: 13/219,661

(22) Filed: Aug. 27, 2011

Related U.S. Application Data
(60) Provisional application No. 61/404,059, filed on Aug. 27, 2010.

(51) Int. Cl. A61F 13/72; A41B 9/02

(52) U.S. Cl. 604/396; 2/403

(57) ABSTRACT

The present invention relates to an adaptive clothing boxer short undergarment which is suitable for a male or female wearer. More specifically, the present invention relates to a boxer short undergarment directed to facilitating the effective and efficient changing of an individual's diaper by allowing the boxer short to be easily removed. In one embodiment the boxer short undergarment has detachable left and right side seams which enable quick changing of the diaper which is underneath the boxer short undergarment. In yet another embodiment, the inside seam is also detachable allowing complete separation from the front and back portions of the short. In addition, a peek panel or translucent patch may be integrated into the shorts to allow the caregiver quick and easy visual identification as to the soiled status of the diaper without having to disrupt the individual by physically moving them.

### CROSS-REFERENCE TO RELATED APPLICATIONS

This application claims the benefit under Title 35 United States Code §119(e) of U.S. Provisional Patent Application Ser. No. 61/404,059; Filed: Aug. 27, 2010, the full disclosure of which is incorporated herein by reference.

### STATEMENT REGARDING FEDERALLY SPONSORED

### RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates to an adaptive clothing boxer short undergarment which is suitable for a male or female wearer. More specifically, the present invention relates to a boxer short undergarment directed to facilitating the effective and efficient changing of an individual's diaper.

### BACKGROUND OF THE INVENTION

Without limiting the scope of the disclosed invention, the background is described in connection with a novel approach for an effective and efficient changing of an individual's diaper.

Currently, when an individual is unable to care for themselves, they often rely on others for their personal care. Part of this care involves changing and cleaning the individual after they urinate or have a bowel movement. To assist in this process an individual will often wear diapers so as to not soil their clothes and also to help facilitate to changing and cleaning process.

However, due to being unable to care for themselves and the difficulties with changing the diaper, the individual is often only dressed in the diaper and is thus exposed. Attempts to clothe the individual only make the changing process more difficult. If the individual needs to be changed, their clothing has to be slid down the length of their legs and removed, the diaper removed, the individual cleaned, a new diaper fitted, and then their clothing put back on in the same tedious and cumbersome process. For a caregiver this is quite a tedious effort to perform on an adult. Not only is the changing process more difficult, but just checking to see if the individual needs to be changed is cumbersome and tedious for the care provider as well as the individual receiving the care. You often have to adjust the individual's clothing and adjust the actual individual to visually check the soil status of the diaper.

The state of the current process and those items available for this effort are quite tedious and cumbersome and lead to the individual feeling exposed and humiliated. Therefore, there exists a need for a better method and approach to maintain an individual's dignity while being cared for and allowing effective and efficient changing of them.

The present invention proposes a novel clothing article for an effective and efficient changing of an individual's diaper.

### BRIEF SUMMARY OF THE INVENTION

The present invention, therefore, provides a novel adaptive clothing boxer undergarment for an effective and efficient changing of an individual's diaper.

More specifically, the present invention relates to a boxer short undergarment directed to facilitating the effective and efficient changing of an individual's diaper by allowing the boxer short to be easily removed. In one embodiment the boxer short undergarment has detachable left and right side seams which enable quick changing of the diaper which is underneath the boxer short undergarment. In yet another embodiment, the inside seam is also detachable allowing complete separation from the front and back portions of the short. In addition, a peek panel or translucent patch may be integrated into the shorts to allow the caregiver quick and easy visual identification as to the soiled status of the diaper without having to disrupt the individual by physically moving them.

In summary, the present invention discloses a novel adaptive clothing boxer short undergarment. More specifically, the present invention relates to a boxer short directed to facilitating the effective and efficient changing of an individual's diaper.

More specifically, by extension, the disclosed short can be used in any environment or case where a short is needed to be removed effectively and efficiently.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is an anterior view of the adaptive clothing boxer short undergarment in accordance with embodiments of the disclosure;

FIG. 2 is an anterior view of the adaptive clothing boxer short undergarment illustrating the right velcro seam being detached in accordance with embodiments of the disclosure;

FIG. 3 is a magnified anterior view of the adaptive clothing boxer short undergarment illustrating the right velcro seam being detached and the fold of the back velcro strip in accordance with embodiments of the disclosure;

FIG. 4 is an anterior view of the adaptive clothing boxer short undergarment illustrating both velcro seams being detached along with the inner velcro strips used to attach the padding in accordance with embodiments of the disclosure;

FIG. 5 is a anterior view of the adaptive clothing boxer short undergarment with an integrated front lift patch in accordance with embodiments of the disclosure.

### DETAILED DESCRIPTION OF THE INVENTION

Disclosed herein is a novel adaptive clothing boxer undergarment. The numerous innovative teachings of the present invention will be described with particular reference to several embodiments (by way of example, and not of limitation).

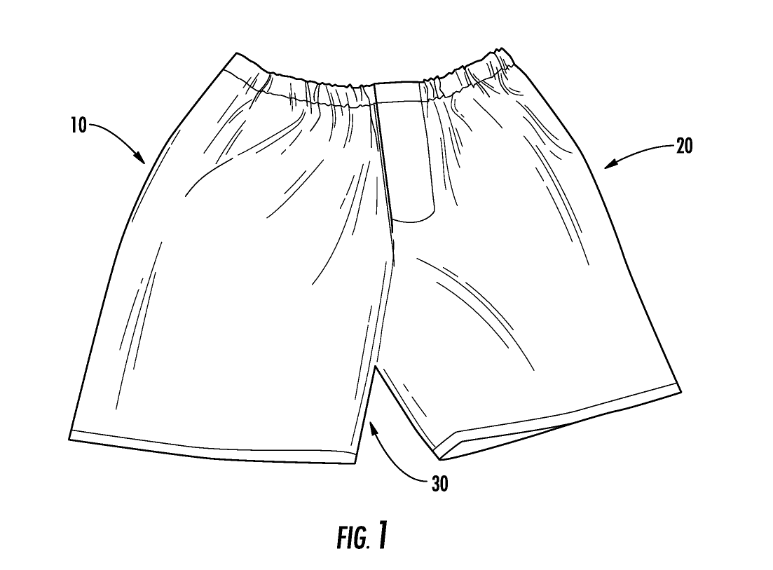

Reference is first made to FIG. 1, an anterior view of the adaptive clothing boxer short undergarment. In this illustration the right seam (10), left seam (20), and inner seam (30) are identified on the boxer short undergarment.

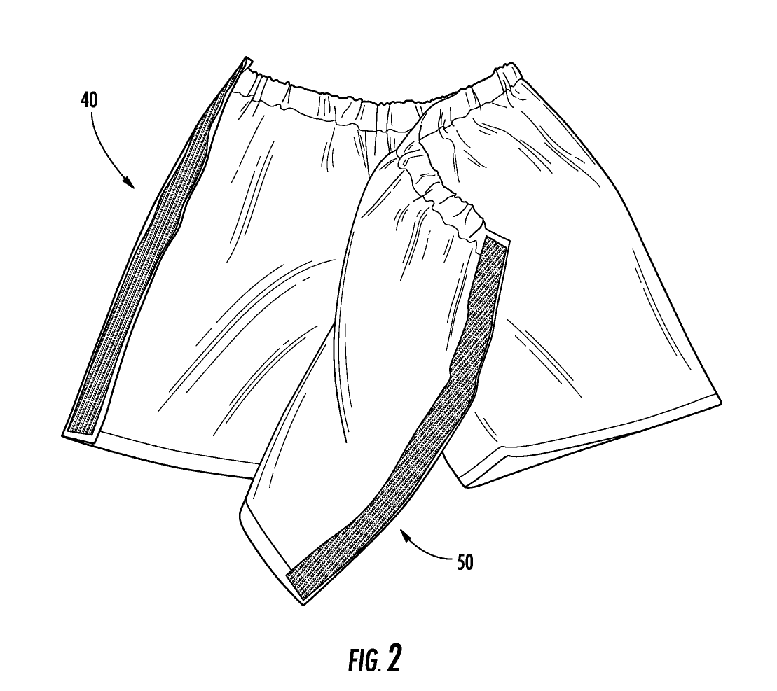

Reference is now made to FIG. 2, an anterior view of the adaptive clothing boxer short undergarment illustrating the right velcro seam (10) being detached and the two inner velcro strips being shown (40)(50). The velcro strip (40) on the back portion of the right seam (10) is shown on the outer layer of that back portion. The velcro strip (50) on the front portion of the right seam (10) is shown on the inner layer of that front portion. This is important as this configuration provides for a smooth inner surface of the right seam for the wearer.

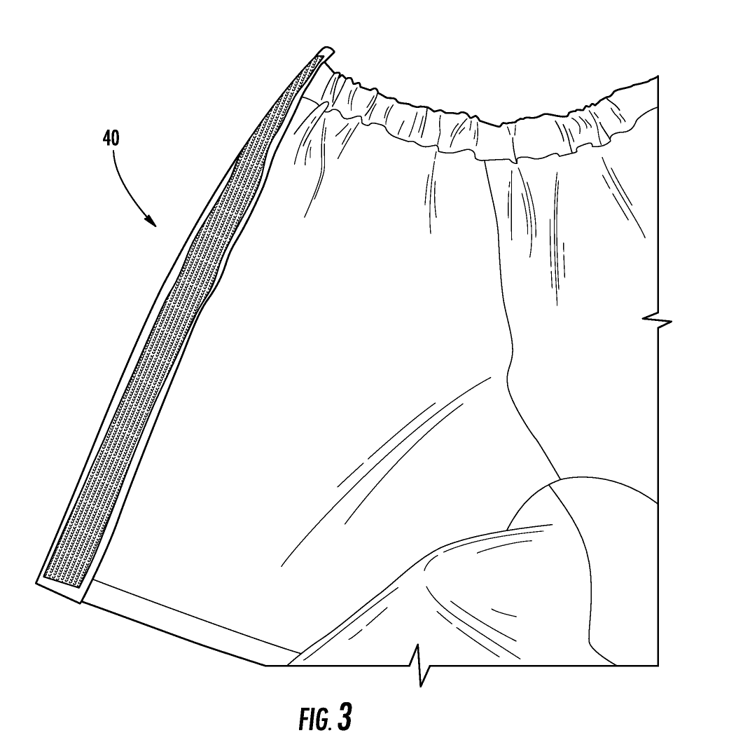

Reference is now made to FIG. 3, a magnified anterior view of the adaptive clothing boxer short undergarment illustrating the right velcro seam (10) being detached and the location of the back velcro strip (40) as previously discussed.

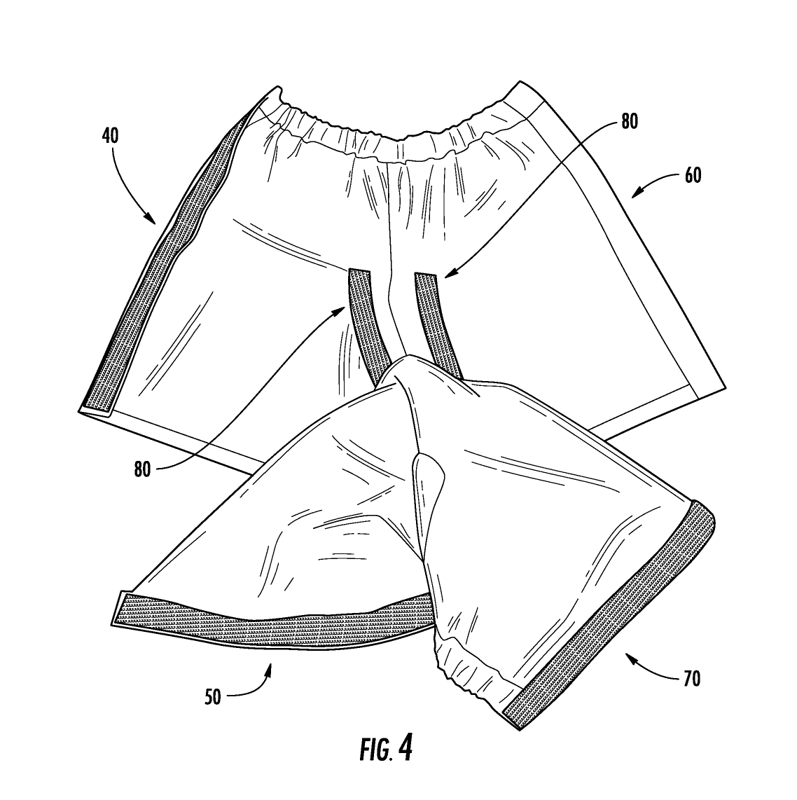

Reference is now made to FIG. 4, an anterior view of the adaptive clothing boxer short undergarment illustrating both velcro seams (10)(20) being detached along with the inner velcro strips (80) used to attach the padding. The configuration of the left seam velcro strips (60)(70) are the same as the left seam velcro strips. That is, the velcro strip (60) on the back portion of the left seam (20) is shown on the outer layer of that back portion. The velcro strip (70) on the front portion of the left seam (20) is shown on the inner layer of that front portion. The inner velcro strips (80) are used to attach the diaper to the shorts which enables the integration of the short with the diaper. Velcro strips would be applied to the diaper to allow the attaching to the shorts.

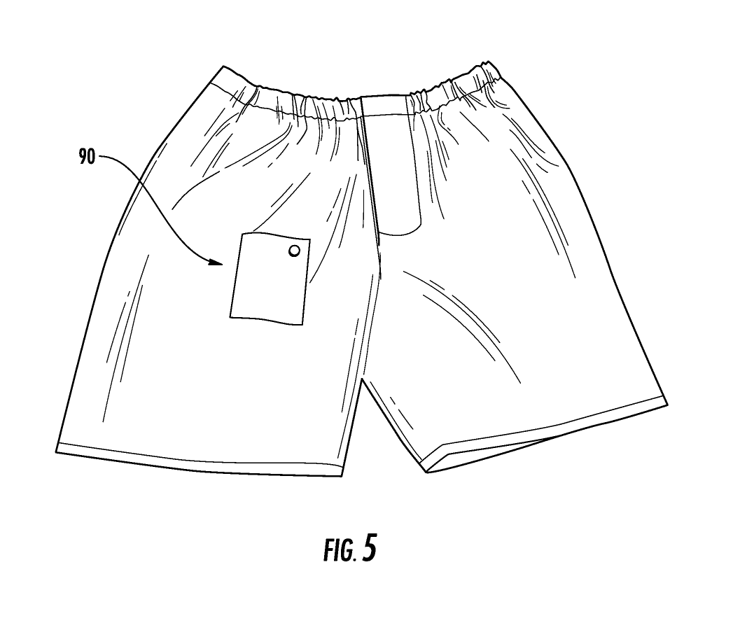

Reference is now made to FIG. 5, a anterior view of the adaptive clothing boxer short undergarment with an integrated front lift patch (90). This front lift patch (90) provides a means for seeing through the shorts to the diaper to verify the soil status of the diaper. This patch (90) may be closed with a button as illustrated in the figure or with other attachment means such as but not limited to velcro. In yet another embodiment, the integrated front lift patch (90) is made of a translucent material to allow for a means of seeing through. While velcro has been described in providing for a means of attaching and deattaching the left seam (10), right seam (20, inner seam (30), and diaper inner attachment strips (80) other attaching means may be employed such as but not limited to buttons, zippers, tapes, and adhesives.

In brief, the adaptive clothing boxer undergarment as described herein provides for an effective and efficient changing of an individuals diaper.

The disclosed invention is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed method, except as may be outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific article described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The invention disclosed and claimed herein can be made and executed without undue experimentation in light of the present disclosure. While this invention has been described in terms of preferred embodiments, it will be apparent to those skilled in the art that variations may be applied to the invention described herein without departing from the concept, spirit, and scope of the invention.

More specifically, it will be apparent that certain components which are both shape and material related may be substituted for the components described herein while the same or similar results would be achieved. All such similar substitutes and modifications apparent to those skilled in the art are deemed to be within the spirit, scope, and concept of the invention as defined by the appended claims.

I claim:

1. A short, comprising: a short which contains means for detaching and reattaching the left and right side seams.

2. The short of claim 1, wherein said short is a boxer undergarment short.

3. The short of claim 1, wherein said short has a means for seeing through said short to identify the soil status of the diaper underneath.

4. The short of claim 2, wherein said short has a means for seeing through the short to identify the soil status of the diaper underneath.

5. The short of claim 1, wherein said short has a means for attaching a diaper as a liner to the inner portion of said diaper.

6. The short of claim 2, wherein said short has a means for attaching a diaper as a liner to the inner portion of said diaper.

7. The short of claim 3, wherein said short has a means for attaching a diaper as a liner to the inner portion of said diaper.

8. A short, comprising: a short which contains means for detaching and reattaching the left side, right side, and inner seams.

9. The short of claim 8, wherein said short is a boxer undergarment short.

10. The short of claim 8, wherein said short has a means for seeing through said short to identify the soil status of the diaper underneath.

11. The short of claim 9, wherein said short has a means for seeing through the short to identify the soil status of the diaper underneath.

12. The short of claim 8, wherein said short has a means for attaching a diaper as a liner to the inner portion of said diaper.

13. The short of claim 9, wherein said short has a means for attaching a diaper as a liner to the inner portion of said diaper.

14. The short of claim 10, wherein said short has a means for attaching a diaper as a liner to the inner portion of said diaper.
